(19) United States
(12) Patent Application Publication
(10) Pub. No.: US 2002/0169460 A1
(43) Pub. Date: Nov. 14, 2002

(54) Head support

(76) Inventors: Mark Leighton Foster, Victoria (CA); Scott Howard Phillips, Victoria (CA); Paul Wesley Taylor, Sidney (CA); Adrian Albert Van Wijk, Vancouver (CA); Dan Reinstein, London (GB)

Correspondence Address: Ralph A Dowell, Dowell & Dowell, 1215 Jefferson Davis Highway Suite 309, Arlington, VA, 22202-3124, US

(21) Appl. No.: 09/914,925

(22) PCT Filed: Jan. 5, 2001
(86) PCT No.: PCT/CA01/00009

(30) Foreign Application Priority Data
Jan. 6, 2000 (CA) 2.295.431
Feb. 24, 2000 (CA) 2.299.483

(51) Int. Cl. A61B 19/00

(52) U.S. Cl. 606/130

(57) ABSTRACT

The invention provides a head support apparatus. The apparatus may for example be used for ophthalmic examination. In some embodiments, the apparatus may allow six degrees of freedom of motion during initial positioning of a patient's head, and may then be locked to hold the patient's head in place precisely. The head support mechanism may include three shafts connecting an upper frame to a structure. Each shaft may be provided with a ball-and-socket joint at one end and a lockable bearing assembly on the other end. The lockable bearing assembly may provide a combination of sliding and pivotal movement. A biasing assembly may be provided to support each shaft, and to return each shaft to a self-centered and fully lifted rest position. A head clamping apparatus is also disclosed comprising pivoting clamping arms and an adjustable chin rest.

### FIELD OF THE INVENTION

The invention is in the field of apparatus used in the inspection and treatment of medical conditions in human patients. The apparatus of the invention may be adapted to be mounted in a fixed position, such as on a table, where it may be used to accommodate a patient's head to support, position and stabilize the head. The apparatus may for example be used for surgical or diagnostic procedures.

### BACKGROUND OF THE INVENTION

Many medical procedures require a patient's head to be positioned precisely. A wide variety of innovative supporting frames for immobilizing a patient's head have therefore been adapted to meet the particular needs of different procedures (as disclosed for example in U.S. Pat. Nos. 5,591,175 issued to Juto Jan. 7, 1997; 5,702,406 issued to Vilsmeire et al. Dec. 30, 1997; 5,337,760 issued to Nichols Aug. 16, 1994; and 5,010,898 issued to de Kanawati et al. Apr. 30, 1991).

Where a patient's head must be positioned to interact with a surgical or diagnostic device, one option is to provide a fixed frame for immobilizing the patient's head in a predetermined position with respect to the instrument. This may however, require a patient to adopt an uncomfortable position in order to adapt to the fixed frame. There is a need, particularly for lengthy procedures in which a patient is to remain conscious, to provide an immobilizing frame that may easily be adapted to a position that is both comfortable for the patient and convenient for the procedure. In some procedures, it may also be desirable to be able to quickly fix the position of a patient's head, once a position has been found that is optimal. While it may be desirable to quickly fix a patient's head in position, this is preferably accomplished without suddenly engaging the patient's head in a manner that would cause discomfort or cause claustrophobia for the patient. It may also be desirable to afford an opportunity in some circumstances to reposition a patient's head after it has initially been fixed in position, either to adopt a new position for an alternative procedure or in the event that the position that is initially fixed is subsequently determined to be suboptimal.

In various embodiments, it is an object of the present invention to provide an apparatus to support, position or immobilize a patient's head to facilitate surgical or diagnostic procedures, including procedures such as opthalmic ultrasonography, neuro-imaging or stereotactic neurosurgical treatment, in which a patient's head may be positioned and then immobilized.

### SUMMARY OF THE INVENTION

In one aspect, the invention provides a head support comprising (including but not limited to): a) an upper frame adapted to accommodate a patient's head; b) one or more shafts connected to the upper frame and extending outwardly from the frame, so that there is a distal portion of the shaft some distance from the upper frame; and, c) a lockable bearing assembly connecting the distal portion of the shaft to a structure, the lockable bearing assembly being constructed to afford the shaft freedom of axial and pivotal motion when in an unlocked configuration and locking the shaft against motion when in a locked configuration. In one embodiment, three shafts may be disposed radially about the upper frame to form a tripod connection between the upper frame and the fixed structure. The shaft may be connected to the upper frame by a joint, such as a ball-and-socket joint, that permits pivotal movement of the shaft with respect to the frame, affording the frame freedom of rotational movement about three perpendicular axes with respect to the shaft. The fixed structure may be a lower frame member adapted for mounting the head support of the invention to an external surface. The upper frame may be adapted to engage a head immobilization apparatus adapted to be secured to the patient's head in a fixed orientation.

The lockable bearing assembly connecting the shaft to the fixed structure may comprise a split ball having spaced apart spherical segments, i.e. portions of a sphere, such as hemispherical segments, the distal portion of the shaft being enclosed by the segments of the split ball. A ball clamp may be provided, enclosing the split ball, and the ball clamp may be adapted so that it may be actuated to releasably compress the split ball on the shaft to lock the shaft against motion with respect to the lockable bearing. The ball clamp may further comprise two or more clamping members that are operable about a hinged connection to compress the spaced apart spherical segments of the split ball. The spaced apart spherical segments of the split ball, such as the hemispherical halves of a split ball, may be spaced apart along a plane that is aligned to permit compression of the hemispherical halves by the clamping members, i.e. movement of the spherical segments towards one another to reduce the distance by which they are spaced apart. The operational orientation of the split ball and the clamping members which facilitates this clamping motion may be maintained by the engagement of a pin on at least one of the clamping members with a slot in at least one of the spherical segments.

A biasing assembly may be used to connect the shaft to the lockable bearing assembly, so that the biasing assembly biases the shaft into a set resting position in the absence of external force on the shaft when the lockable bearing is in the unlocked configuration. In such an embodiment, the head support of the invention is returned to a pre-set resting position when not in use. In some embodiments, the biasing assembly may comprise at least three extension springs connecting the shaft to the lockable bearing assembly. The distal ends of the springs may be radially disposed about the shaft by attachment to a bottom spring plate attached to the shaft. The bottom spring plate may for example be attached to the bottom end of the shaft. The proximal ends of the springs may be radially disposed about the lockable bearing assembly by attachment to a top spring plate, the top spring plate being fixed to the lockable bearing assembly. Each of the springs in the spring biasing assembly may be housed in a rigid spring sheath, such as a rigid tube, so that the springs bias the bottom spring plate into engagement with the spring sheaths in the resting position. In this way, the spring sheaths may be used to form a tripod support between the top and bottom spring plates, the positioning of the spring sheaths will therefore determine the resting orientation of the upper frame.

In an alternative adaptation, the upper frame may include two pivoting head clamping arms adapted to cup the sides of a patient's head. Left and right pivoting clamping arms may be pivotably connected around shafts in the upper frame assembly. This clamping assembly may further include an adjustable chin rest. The clamping arms may be actuated by the weight of a patient's head, to cradle the head, rather than locking the head into position. As such, the clamping apparatus may be adapted to avoiding causing feelings of claustrophobia. Biasing elements such as one or more springs may be provided to rotate the clamping arms out into their most open position when the apparatus is not in use. A mechanism, such as a cam slot and pin, may be provided to facilitate symmetrical motion of the two clamping arms. The apparatus may thereby be adapted to articulate the clamping arms so that they move symmetrically, which may have the effect of minimizing the motion of the patient's head. It will be appreciated that this design may be adapted to facilitate comfortable, self-adjusting head immobilization on a wide range of head sizes, with little or no need for adjustment of the apparatus between patients.

### BRIEF DESCRIPTION OF THE DRAWINGS

FIG. 1 is an isometric view of a head support of the invention, showing the unmounted support from slightly below the front left hand side of the support

FIG. 2A is an isometric view of a lower spherical bearing, showing a broken away portion of the shaft that is housed in the bearing.

FIG. 2B is a top cross-sectional view of a lower spherical bearing, showing a hinged ball clamp enclosing a split ball and shaft.

FIG. 3 is a side elevational view of a ball and socket joint connecting a shaft to the upper frame, showing the ball socket blocks in cross-section.

FIG. 4 is an isometric view of a lower spherical bearing, showing in phantom the springs inside the spring assembly.

FIG. 5 is an isometric view of a head clamping system with two clamping arms and an adjustable chinrest

FIG. 6A is a plan view of a head clamping system of the invention, with clamping arms engaged on a relatively large head

FIG. 6B is a plan view of a head clamping system with clamping arms engaged on a relatively small head

FIG. 7A is a plan view of a head clamping system showing a cam for providing symmetrical clamping arm motion

FIG. 7B is an isometric view, partially broken away, of a head clamping system showing a cam for providing symmetrical clamping arm motion

### DETAILED DESCRIPTION OF THE INVENTION

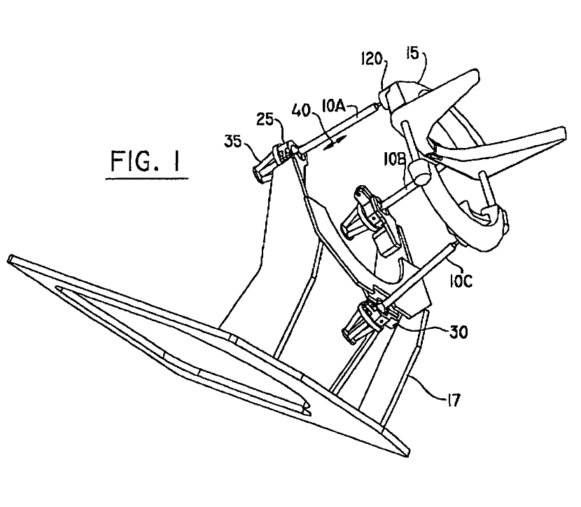

In the embodiment shown in FIG. 1, an upper frame 15 is supported on its periphery by three radially disposed shafts 10A, 10B, and 10C. The use of three shafts 10A, 10B, and 10C provides a tripod-like arrangement, alternative embodiments may use any number of such shafts. Upper frame 15 may be a generally rigid structure, which connects shafts 10A, 10B and 10C. Upper frame 15 may itself be adapted to be securable to a patient's head in a fixed orientation. Alternatively, upper frame 15 may be adapted for mounting to a head immobilization apparatus which is in turn securable to a patient's head in a fixed orientation.

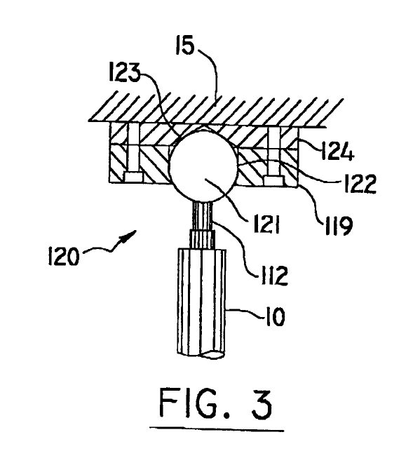

As illustrated, shafts 10A, 10B, and 10C are connected to upper frame 15 by means of spherical joints, such as ball and socket joints 20A, 20B, and 20C. The illustrated ball-and-socket joints each allow three rotational degrees of freedom of the shaft with respect to upper frame 15. As shown in FIG. 3, the top portion of shaft 10 may terminate in a constricted neck region 112 to facilitate angular movement of the shaft. The shaft may be topped with a precision ground ball 121, which is preferably a strong stable bearing material such as an acetal resin (made from acetal homopolymers or acetal copolymers, also known as polyacetals). Socket elements 119, 124 may be attached to upper frame 15, so that inner socket block 124 provides an inner socket surface 123, and outer socket block 119 provides outer socket surface 122. In use, socket surfaces 122, 123 act to retain ball 121 in place and provide bearing surfaces for rotation of ball 121. Socket block 119 and 124 may for example be made from aluminum. In alternative embodiments, the connection between a shaft 10 and frame 15 may be more or less constrained in the permitted range of motion, including solid immovable connections (such as welded or unitary connections) or connections that additionally permit movement in the direction of the axis of the shaft (axial movement).

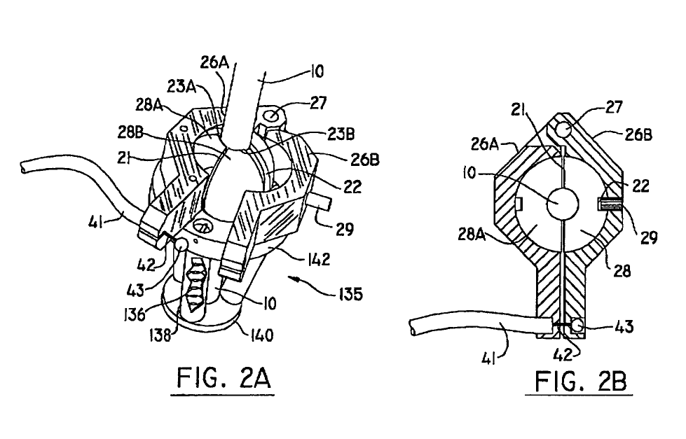

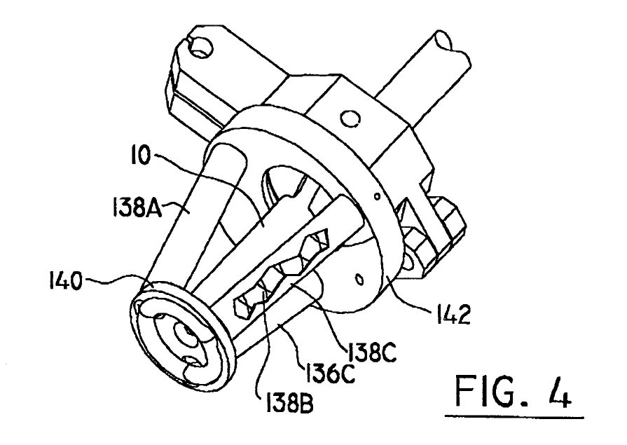

Shafts 10A, 10B, and 10C connect upper frame 15 to a structure, such as a fixed structure (a structure that may be fixed in place), shown as lower frame 17 in the illustrated embodiment, where lower frame 17 may be adapted for fixed mounting to an external surface, such as a table. In the illustrated embodiment, each of the three shafts 10A, 10B, and 10C passes through a lower spherical bearing 25A, 25B, 25C. In the illustrated embodiment, lockable bearing assemblies 25A, 25B, 25C are constructed so as to permit three rotational degrees of freedom of movement of a shaft with respect to lower frame 17, as well as axial motion of the shaft shown by arrow 40. In alternative embodiments, the connection between a shaft and lower frame 17 may be more or less constrained in the range of motion permitted to the shaft, including solid immovable connections or ball-and-socket joints. A biasing assembly 35 may be provided to lift the head support and apply a light upward force to counteract the weight of the head, illustrated in detail in FIGS. 2A and 4.

It will be appreciated that equivalent head support structures of the invention may be provided using a range of shaft connections between upper frame 15 and a structure such as lower frame 17. For example, the illustrated arrangement of connections could be reversed, with ball-and-socket-joints provided on the ends of the shafts that engage lower frame 17, and spherical bearings provided on the ends of the shafts that engage upper frame 15.

As shown in FIGS. 2A and 2B, lockable bearing assembly 25 comprises hinged ball clamp members 26A and 26B which have spherical inner bearing surfaces . The inner surfaces of hinged ball clamp members 26A and 26B are mateable with split ball hemispheres 28A, 28B to constrain axial and pivotal motion of shaft 10, locking shaft 10 in position. The split ball may alternatively be formed from differently arranged spherical segments, such as a ball segmented into three or more segments. Split ball 28A, 28B may be made of a material which is smooth enough for easy axial sliding motion of the shaft when the lockable bearing is in an unlocked configuration, while the material provides sufficient friction to lock shaft 10 against motion when the lockable bearing assembly is in the locked configuration. Split ball 28A, 28B may for example be constructed of an acetal resin.

As illustrated, each hemispherical half of split ball 28A, 28B defines a semicircular slot 23A, 23B through which shaft 10 may slide when the mechanism is unlocked. A gap 21 may be left between the two spaced apart ball halves 28A, 28B such that ball halves 28A, 28B may move towards one another when clamping members 26A, 26B are actuated, although it may be preferred that the ball halves 28A, 28B do not touch when clamped together over shaft 10. Slot 22 may be provided in a ball segment 28B in which pin 29 on clamping member 26B may ride to prevent ball segments 28A, 28B from rotating about shaft 10 and reducing the available clamping force on shaft 10.

In the illustrated embodiment, lockable bearing assembly 25 is clampable by means of a cable actuator mechanism, as shown in FIG. 2A, comprising cable end 43, cable 42 and cable sheath 41. Clamping actuation takes place when the cable 42 is pulled relative to the cable sheath 41 which brings cable end 43 to bear in the bottom of a housing formed in ball clamp member 26B and the sheath to bear on the bottom of a housing formed in ball clamp member 26A. To achieve the locked configuration, hinged ball clamp members 26A and 26B are drawn together about hinge 27 to compress split ball halves 28A, 28B onto shaft 10 so that no rotation of ball 28A, 28B or sliding of the shaft 10 may occur.

As shown in FIGS. 2A and 4, a biasing assembly 35 may be used to connect the shaft to the lockable bearing assembly 25 to modulate movement of shaft 10 with respect to lower frame 17. In the illustrated embodiment, the distal ends of three extension springs 136 are arrayed at approximately 1200 intervals around a bottom spring plate 140 that is attached to the bottom end of shaft 10. The proximal ends of each spring may be attached to a top spring plate 142 which is fixed relative to lockable bearing assembly 25 In the illustrated embodiment, around each spring is a rigid tubular spring sheath 138A, 138B, 138C attached to top spring plate 142. Spring sheaths 138A, 138D, 138C act to limit the compression of each spring 136 at a fixed length. In use, biasing assembly 35 acts to return each shaft 10 to a rest position fully lifted and self-centered when the patient's head is removed from the head support. Equivalent biasing assemblies may be provided using resilient members as alternatives to springs 136. Similarly, spring sheaths 138A, 138B, 138C may be replaced with adjustable rigid spacers that act to determine the resting position of the shafts. Top and bottom spring plates may be integral functional components of a lockable bearing assembly and shaft respectively, or may be separate physical elements as shown in the illustrated embodiment

In some embodiments, if for example the head support apparatus is to be used in an angled attitude as shown in FIG. 1, additional upper frame biasing elements shown as springs 260A, 260A, 260C may be used to resist ‘drooping’ of the upper frame 15 when it is not in use. Upper frame biasing elements 260A, 260B, 260C may be long extension springs, as illustrated, arranged in generally perpendicular orientation with respect to respective shafts 10A, 10B, 10C.

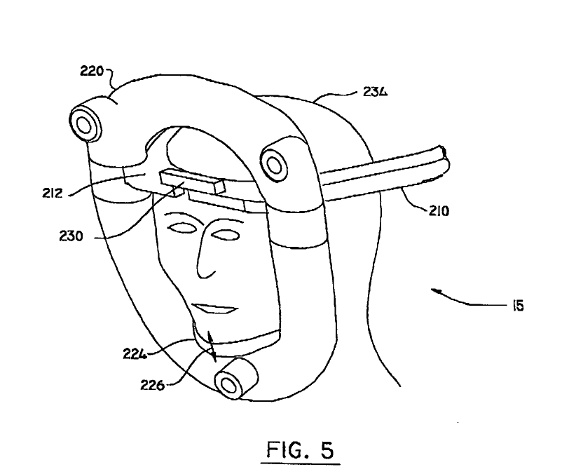

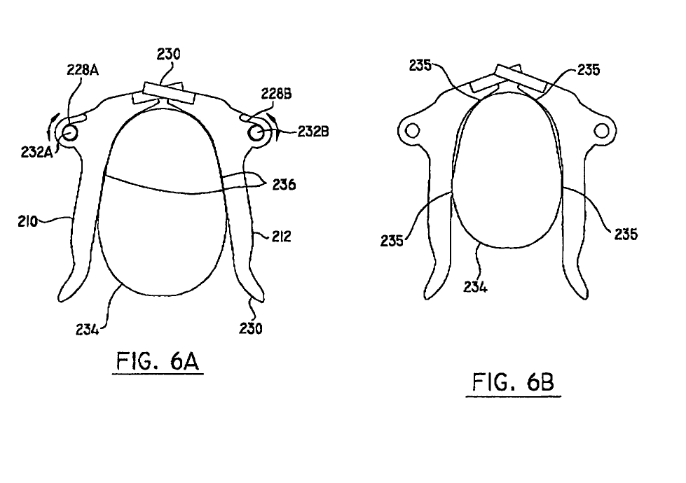

FIG. 5 shows an adaptation of the upper frame 15 to facilitate immobilization of patient's head 234. As illustrated, left clamping arm 210 and a right clamping arm 212 may be pivotably mounted on upper frame 15 so that when patient's head 234 is moved into the head clamping apparatus, frontal portions of the patient's head 234 exert pressure on front portions of left and right clamping arms 210, 212 to pivot clamping arms 210, 212 so that rear portions of clamping arms 210, 212 come into engagement with parietal or occipital portions of patient's head 234, whereby the patient's head is engaged by front and rear portions of each clamping arm. As illustrated, the motion of left and right clamping arms 210, 212 is about pivot centers 232A and 232B, shown as shafts in FIG. 6. In one embodiment, clamping arm biasing elements 228A and 2281, shown as coil springs, may provide sufficient torque to open arms 210, 212 as a patient's head is retracted from the apparatus. In some embodiments, the force exerted by the arm biasing elements will be balanced with the forces exerted on upper frame 15 by biasing assembly 35, so that clamping arms 210, 212 are able to engage head 234 before upper frame 15 is significantly displaced with respect to biasing assembly 35, so that head 234 may be secured by clamping arms 210, 212 prior to manipulation of head 234 into an optimal position for immobilization. Chin support 224 may be provided, which may be vertically adjustable as shown by arrow 226 to compensate for patient chin-to-brow distance variations.

In some embodiments, the apparatus may be largely self-adjusting to accommodate a wide range of head sizes. As shown for example in FIG. 6A, on a relatively large head, arms 210, 212 rest alongside the lateral portions of the head 236, providing even support. As shown in FIG. 6B. on a smaller head 234, clamping arms 210, 212 may be adapted to rotate further to make contact with four points 235 on head 234 to provide good support. Rear clamping arm ends 238 may have a flared shape, as illustrated, in order to guide a patient's head 234 into place, and to reduce any poking hazard to the eye of the patient.

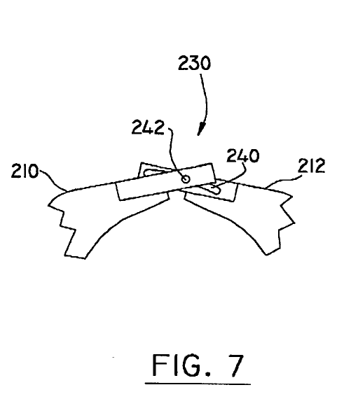

The apparatus of the invention may be adapted for symmetrical motion of clamping arms 210, 212. For example, a motion guide 230 may be included which articulates the arms to rotate equally. Many possible mechanisms may be used to achieve this function. For example, FIG. 7 shows a motion guide mechanism comprising a cam slot 240 mounted on arm 212 and follower pin 242 mounted on arm 210 and engaged in slot 240, so that slot 240 may be adapted to force arms 210, 212 into symmetrical motion.

In one embodiment, the head support of the invention may be adapted for use in the field of ophthalmological ultrasonography. In ophthalmological ultrasonography, there is generally a need to allow a patient's head to move so that the patient's eye is in a suitable position for ultrasound scanning, and then to keep it the patient's head from moving during an ultrasound scan. Ophthalmological ultrasonography also generally requires a patient's eye to be immersed in a fluid bath, where the fluid mediates the transmission of sound from the transducer to the eye. The head support of the present invention may therefore be adapted to permit a patient to look down into an ultrasonography device, at an angle that facilitates engagement with an eye cup, with an eye seal of the eye cup surrounding the orbit of the eye to isolate a volume of fluid in the region of the eye. Utilizing the frame of the invention, the patient's head may be moved into position, allowing a seal to be made around the orbit in a position in which the eye is aligned with the optics of the ultrasound machine. In some embodiments, the apparatus may allow six degrees of freedom of motion within a defined range of space during initial positioning of a patient's head. The apparatus of the invention may then be used to permit the patient's head to be fixed in position for the selected medical procedure.

Although various embodiments of the invention are disclosed herein, many adaptations and modifications may be made within the scope of the invention in accordance with the common general knowledge of those skilled in this art Such modifications include the substitution of known equivalents for any aspect of he invention in order to achieve the same result in substantially the same way. Numeric ranges are inclusive of the numbers defining the range. The word “comprising” is used as an open-ended term, substantially equivalent to the phrase “including, but not limited to”. In some embodiments, adaptations of the invention may be provided so that a patient's head it may be possible to immobilize a patient's head to a submicronic level of stability, comfortably and without causing psychological distress.

What is claimed is:

1. A head support comprising:
a) an upper frame adapted to accommodate a patient's head;
b) a shaft connected to the upper frame and extending outwardly from the frame to a distal portion of the shaft; and,
c) a lockable bearing assembly connecting the distal portion of the shaft to a structure, the lockable bearing assembly affording the shaft freedom of axial and pivotal motion when in an unlocked configuration and locking the shaft against motion when in a locked configuration.

2. The head support of claim 1, wherein the lockable bearing assembly comprises:
a) a split ball having spaced apart spherical segments, the distal portion of the shaft being enclosed by the segments of the split ball; and,
b) a ball clamp enclosing the split ball and adapted for actuation to releasably compress the split ball on the shaft to lock the shaft against motion with respect to the lockable bearing.

3. The head support of claim 2, wherein the ball clamp comprises clamping members that are operable about a hinged connection to compress the spaced apart spherical segments of the split ball.

4. The head support of claim 3, wherein the split ball has two spaced apart hemispherical halves, the hemispherical halves being spaced apart along a plane that is aligned to permit compression of the hemispherical halves by the clamping members, the orientation of the split ball and the clamping members being maintained by the engagement of a pin on at least one of the clamping members with a slot in at least one of the hemispherical halves.

5. The head support of claim 4, farther comprising a biasing assembly connecting the shaft to the lockable bearing assembly, biasing the shaft into a set Testing position in the absence of external force on the shaft when the lockable bearing is in the unlocked configuration.

6. The head support of claim 5, wherein the biasing assembly comprises at least three extension springs connecting the shaft to the lockable bearing assembly, the distal ends of the springs being radially disposed about the shaft by attachment to a bottom spring plate attached to the shaft, the proximal ends of the springs being radially disposed about the lockable bearing assembly by attachment to a top spring plate fixed to the lockable bearing assembly, each of the springs being housed in a rigid spring sheath, so that the springs bias the bottom spring plate into engagement with the spring sheaths in the resting position wherein the spring sheaths form a tripod support between the top and bottom spring plates.

7. The head support of claim 1, wherein the shaft is connected to the upper frame by a joint that permits pivotal movement of the shaft with respect to the frame, affording the frame freedom of rotational movement about three perpendicular axes with respect to the shaft.

8. The head support of claim 6, wherein the shaft is connected to the upper frame by a joint that permits pivotal movement of the shaft with respect to the frame, affording the frame freedom of rotational movement about three perpendicular axes with respect to the shaft.

9. The head support of claim 6, wherein the shaft is connected to the upper frame by a ball-and-socket joint.

10. The head support of claim 1, comprising three shafts disposed radially about the upper frame to form a tripod connection between the upper frame and the fixed structure.

11. The head support of claim 2, comprising three shafts disposed radially about the upper frame to form a tripod connection between the upper frame and the fixed structure.

12. The head support of claim 6, comprising three shafts disposed radially about the upper frame to form a tripod connection between the upper frame and the fixed structure.

13. The head support of claim 7, comprising three shafts disposed radially about the upper frame to form a tripod connection between the upper frame and the fixed structure.

14. The head support of claim 8, comprising three shafts disposed radially about the upper frame to form a tripod connection between the upper frame and the fixed structure.

15. The head support of claim 1, wherein the structure is a lower frame member adapted for fixed mounting to an external surface.

16. The head support of claim 8, wherein the structure is a lower frame member adapted for fixed mounting to an external surface.

17. The head support of claim 1, wherein the upper frame is adapted to engage a head immobilization apparatus, and the head immobilization apparatus is adapted to be secured to the patient's head in a fixed orientation.

18. The head support of claim 16, wherein the upper frame further comprises a head clamping apparatus, and the head clamping apparatus is adapted to be secured to the patient's head in a fixed orientation.

19. The head support of claim 18, wherein the head clamping apparatus comprises:
a) left and right spaced apart clamping arms disposed to engage opposite sides of the patient's head, the arms being pivotably mounted to the upper frame so that frontal portions of the patient's head may exert pressure on front portions of each clamping arm to pivot the clamping arms so that rear portions of the clamping arms come into engagement with parietal or occipital portions of the patient's head, whereby the patient's head is engaged by front and rear portions of each clamping arm.

20. A method of ophthalmological ultrasonography comprising:
a) attaching a lockable head support to a patient's head, so that the patient's head has six degrees of freedom of motion within a defined range of space;
b) positioning the patient's head in engagement with an eye seal and providing a fluid within a space defined by the eye seal for transmission of sound between an ultrasound transducer and the patient;
c) locking the patient's head in a fixed position for ultrasonographic examination.
